(19) United States
(12) Patent Application Publication
(10) Pub. No.: US 2005/0251082 A1
(43) Pub. Date: Nov. 10, 2005

(54) Removable cavity wound dressings

(76) Inventor: Michelle Del Bono, Barnoldswick (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/480,786

(22) PCT Filed: Jun. 20, 2002
(86) PCT No.: PCT/GB02/02809

§ 371 Date: Jun. 21, 2004

(30) Foreign Application Priority Data
Jun. 21, 2001 (GB) 0115219.8

(51) Int. Cl. A61F 13/00

(52) U.S. Cl. 602/41

(57) ABSTRACT

A packaged wound dressing comprising an elongated, absorbent cavity wound dressing that is packaged in contact with two opposed sheets of microorganism-impermeable plastic sheet material, wherein the opposed sheets contact the dressing to form a microorganism-impermeable barrier around the circumference of the dressing in at least a region of the dressing. The invention also provides a method of making such a wound dressing comprising the steps of: providing an elongated microorganism-impermeable plastic sheet; providing one or more mold cavities in said elongated sheet for molding an elongated wound dressing; introducing a fluid precursor of a solid wound dressing material into said one or more mold cavities; and allowing said fluid precursor to set in said plurality of mold cavities.

The present invention relates to improved cavity wound dressings and to methods for the manufacture thereof.

There is a need in clinical practice for absorbent medicinal articles that can be inserted into deep wounds to absorb wound exudate and provide cavity filling. Such dressings should be highly absorbent, conformable to the shape of the wound, and inexpensive.

It is known to provide textile or foam-based absorbent structures for dressing deep wounds. Those structures are insufficiently conformable to fit every deep wound, unless they are cut into shape. Furthermore, such absorbent structures can be relatively difficult to remove from a deep wound after use without causing further trauma.

U.S. Pat. No. 4,837,285 describes the use for soft tissue cavity filling of resorbable collagen sponge beads having diameters of from 0.1 to 4.0 mm. The beads have pore sizes of from 50 to 350 micrometers. Such beads can be difficult to handle, and are not suitable for use as a removable, absorbent dressing for deep wounds.

DE-A-4037931 describes a deep medicinal article structure consisting of a cavity filling plug of resorbable collagen foam containing an array of hollow resorbable filaments. The filaments are bundled together at one end, and connected to a drain for wound fluid, whereby the wound fluid is continuously drained from the wound cavity through the hollow filaments. Such a structure is relatively expensive to construct, and insufficiently conformable to a wide range of wound cavity shapes.

EP-A-0171268 describes absorbent, non-adherent wound dressings for use in the treatment of deep wounds, wherein the dressing comprises a porous bag containing individual pieces of a conformable, resilient, absorbent hydrophilic foam. The porous bag may be provided with a string to assist removal of the bag from the wound after use.

EP-A-0575090 describes products suitable for absorbing wound exudate comprising a string along which beads of alginic acid or alginate are threaded. U.S. Pat. No. 5,470,625 describes an elongated wound packing product comprising individual linked segments of hydrogel enclosed within a liquid-permeable pliable material. U.S. Pat. No. 3,095,877 describes a surgical packing comprising a hollow nylon thread having a plurality of absorbent balls centrally secured to the thread in spaced relation to each other.

In a first aspect, the present invention provides a packaged wound dressing comprising an elongated, absorbent cavity wound dressing that is packaged in contact with two opposed sheets of microorganism-impermeable plastic sheet material, wherein the opposed sheets contact the dressing to form a microorganism-impermeable barrier around the circumference of the dressing in at least a region of the dressing.

The microorganism-impermeable barrier is typically a physical barrier formed by adhesion of the sheets around the circumference of the dressing. This reduces the need for secondary packaging around the dressing.

In certain embodiments, the opposed sheets contact the dressing to form a microorganism-impermeable barrier around the circumference of the dressing substantially along the whole length of the dressing. Preferably, this results in a substantially encapsulated dressing, i.e a dressing with the packaging sheets tightly enclosing the absorbent material substantially over the whole external surface of the dressing so as to leave minimal air gaps between the sheets and the dressing.

In other embodiments the opposed sheets contact the dressing to form a microorganism-impermeable barrier around the circumference of the dressing at a plurality of intervals along the length of the dressing.

Preferably, the plastic films are bonded together at least in part by means of a releasable adhesive so that the package can be opened by peeling apart the films. Preferably, the packaged dressing comprises a first microorganism-impermeable sheet coated on its inner surface with a pressure-sensitive adhesive, and a second microorganism-impermeable sheet (which may be the same sheet folded over) coated on its inner surface with a release coating, such as silicone or fluoropolymer, for forming a releasable adhesive bond with the pressure-sensitive adhesive.

Preferably, an internal surface of the polymer films that contacts the dressing comprises a non-stick or release material to assist removal of the dressing from the package.

The absorbent wound dressing may be any elongated absorbent body that can be coiled or packed into a cavity wound. Preferably, it comprises an absorbent hydrophilic foam or a hydrogel such as a polyurethane foams and hydrogels as hereinafter described. The length of the dressing is normally at least 10 times its mean width, and more preferably at least 20 times its mean width.

Preferably, the wound dressing comprises a plurality of absorbent bodies linked by one or more flexible bodies as hereinafter described. Preferably, the absorbent body has been formed in situ between the two sheets of plastic material, for example by injecting a foamable polyurethane precursor composition into a tubular cavity or mold between the sheets.

The invention provides an advantage that the cavity wound dressing can be manufactured in indefinite lengths, for example 50 cm or 1 m or 10 m or more, for example in rolls, without any need for sterile secondary packaging. The lengths of rolls are straightforward to use, and lengths are simply cut to provide a sufficient volume of cavity filling material to pack any given wound. The fact that the packaging sheet contact the dressing at least at intervals along the length of the dressing allows lengths to be cut from the dressing without the remainder of the dressing becoming contaminated, and still without the need for secondary packaging.

Preferably, the mean cross sectional area of the elongated absorbent body or bodies in the uncompressed and unhydrated state is from 1 mm² to 100 mm², preferably from 5 mm² to 25 mm². Preferably, the mean linear density of the absorbent material in the unhydrated state is from 1 g/m to 100 g/m, preferably about 5 g/m to about 15 g/m.

The absorbent body or bodies may be made of any material that absorbs wound fluid and is acceptable for application to a wound, including woven and nonwoven textile materials, biopolymers such as alginates and cellulose, and materials that are fully absorbable in vivo such as collagen sponges. Preferably, the materials are not fully absorbable in vivo, since it is a particular advantage of the present invention that it allows easy removal of wound dressings after use. Preferably, the materials will absorb at least three times their weight of wound fluid, and more preferably at least four times, as determined by immersing the materials in physiological saline at 25° C. for 60 minutes, draining without compression, and weighing.

Preferably, the absorbent body or bodies comprise a hydrophilic foam or a hydrogel. Such materials are less prone to shed particles into the wound cavity than are textile materials.

Suitable hydrophilic foam bodies have a density of from 0.28 to 0.5 g/cm³, and more preferably from 0.32 to 0.48 g/cm³. Preferably, the foam has an elongation to break of at least 150%, more preferably from 500% to 1000%. The foam is hydrophilic and absorbs aqueous fluids such as wound exudate with swelling. However, the foam is preferably highly cross-linked and substantially insoluble in water. Preferably, the foam has an absorbency of at least 3 g saline/g, and preferably a swellability in water of at least 200%. Preferably, the foam is an open-cell foam.

Preferably, the hydrophilic foam comprises less than 10% water prior to use as an absorbent, more preferably less than 5% water and more preferably it contains less than 2% of water before use.

Suitable foams include polyurethane foams, carboxylated butadiene-styrene rubber, polyacrylate, polyvinylic or cellulosic foams. Preferably, the foam comprises a polyurethane. Preferably, the foam polyurethane is formed by crosslinking an isocyanate-capped prepolymer. Preferably, the prepolymer comprises an isocyanate-capped polyether prepolymer, and more preferably it comprises an isocyanate-capped ethyleneoxy/propyleneoxy prepolymer. For example, one of the prepolymers available under the Registered Trade Mark HYPOL from Dow Chemical Company, 2 Heathrow Boulevard, 284 Bath Road, West Drayton, UK. Preferably, the hydrophilic foam is a polyurethane foam as described in EP-A-0541391, the entire content of which is incorporated herein by reference.

The term “hydrogel” refers to medically acceptable macromolecular substances that form a gel with water under physiological conditions of temperature and pH. Such hydrogels preferably have the ability to swell and absorb fluid while maintaining a strong integral structure. Preferably, the hydrogel composition forms a gel that is substantially insoluble in water under physiological conditions, whereby the hydrogel is not washed away by the wound fluid.

Exemplary insoluble gels include certain cross-linked polyacrylate gels, calcium alginate gels, cross-linked hyaluronate gels, wherein the hydrogel layer comprises a hydrogel material selected from gels formed from vinyl alcohols, vinyl esters, vinyl ethers and carboxy vinyl monomers, meth(acrylic) acid, acrylamide, N-vinyl pyrrolidone, acylamidopropane sulphonic acid, PLURONIC (Registered Trade Mark) (block polyethylene glycol, block polypropylene glycol) polystyrene-, maleic acid, NN-dimethylacrylamide diacetone acrylamide, acryloyl morpholine, and mixtures thereof. Preferably, the gel adheres strongly to the flexible material. In certain embodiments the gel may be chemically bonded to the flexible material.

Preferably, the hydrogel layer comprises a hydrogel material selected from polyurethane gels, biopolymer gels, carboxymethyl cellulose gels, hydroxyethyl cellulose gels, hydroxy propyl methyl cellulose, modified acrylamide and mixtures thereof. Suitable biopolymer gels include alginates, pectins, galactomannans, chitosan, gelatin, hyaluronates and mixtures thereof. Some of these biopolymer materials also promote wound healing. In certain embodiments, the hydrogel layer comprises a hydrogel material of the kind described in WO00/07638, the entire content of which is incorporated herein by reference.

Preferably, the gels are cross-linked, and the cross-linking may be either covalent or ionic.

Preferably, the hydrogel material further comprises from 5 to 50% by weight on a dry weight basis of one or more humectants such as glycerol

It will be appreciated that the absorbent bodies may comprise any combination of textile filaments, hydrogel and/or hydrophilic sponge.

Preferably, the absorbent bodies contain a medicament. Suitable medicaments include antimicrobials, growth factors, steroids, antifungal agents, spermicidal agents, and anaesthetics. Suitable antimicrobials include antibiotics, silver salts, povidone iodine and chlorhexidine. Preferred amounts of the antimicrobials are from 0.01 to 100 mg per gram of absorbent body on a dry weight basis, more preferably from 0.1 to 10 mg/g.

In certain embodiments the absorbent bodies may comprise a therapeutically active biopolymer such as collagen, an alginate or hyaluronic acid (to promote wound healing), or oxidized regenerated cellulose (a haemostat).

In certain embodiments the absorbent bodies may comprise activated charcoal for odor absorption. Optionally the activated charcoal containing strip may include an antimicrobial agent such as iodine or silver. Suitable materials are the silver containing charcoal cloth described in GB-A-2127389 and the elastomeric foam impregnated with charcoal and iodine described in EP-A-0053936.

The embodiments comprising a plurality of linked absorbent bodies provide high conformability to any wound cavity shape or size. The flexible linkage enables the dressing to take up any shape, but also enables the dressing to be removed from the cavity easily just by pulling on any part of the dressing.

Preferably, the plurality of absorbent bodies comprises at least three absorbent bodies, more preferably at least five, and most preferably at least eight absorbent bodies. In certain preferred embodiments, the dressing may be manufactured in lengths of 10, 20, 50, 100 or even more absorbent bodies, in which case the dressing would normally be cut to size by the care giver before application to the wound. This enables different lengths of the same dressing to be used for a variety of differently sized wounds without wastage.

For optimum comfort and conformability the individual linked absorbent bodies preferably each have a volume of from 0.001 to 10 cm³, more preferably from 0.01 to 1 cm³.

Preferably, the individual linked absorbent bodies have an aspect ratio (ratio of the largest dimension to the smallest dimension) no greater than 5, preferably no greater than 3, and more preferably no greater than 2. In certain embodiments the absorbent bodies are substantially round.

The flexible bodies linking the absorbent bodies in these embodiments may be in the form of a flexible strip or sheet. However, for maximum conformability of the wound dressing the one or more flexible bodies comprise a filament, a thread or a string. Preferably, the flexible bodies consist of a single linear filament, thread or string. Preferably, the absorbent bodies are spaced along the filament, thread or string. The dressing then preferably takes the form of an absorbent string of beads.

Suitable materials for the filament, thread or string are preferably highly flexible, medically acceptable and non-biodegradable. Nylon thread is preferred.

Preferably, the individual linked absorbent bodies are spaced at uniform intervals along the filament, thread or string. Preferably, the mean length of filament, thread or string between the absorbent bodies is from 1 to 40 mm, more preferably from 5 to 20 mm.

The flexible body is attached to the absorbent bodies by knotting, adhesion or melt bonding. Preferably, the filament, thread or string extends through the absorbent bodies, more preferably near the center of the absorbent bodies, in order to provide the most secure attachment of the absorbent bodies to the filament, thread or string.

Preferably the cavity wound dressing according to the present invention is sterile, and it may be packaged in a microorganism-impermeable secondary container.

The cavity wound dressing is encapsulated between two sheets of microorganism-impermeable polymer film material. This film material functions as the primary packaging for the dressing, and may remove any need for a secondary outer packaging envelope. It also enables the dressing to be made, stored and used in lengths or rolls. The dressing may be encapsulated between the two sheets of film material by vacuum encapsulation, heat shrinkage, or preferably by forming the absorbent dressing in situ between the two plastic films as described in more detail below. The plastic films may be opposite sides of a plastics film tube, or they may be a single sheet folded over and bonded along a margin, or they may be two separate sheets bonded along opposed margins. The bonding between the sheets may be melt bonding or permanent adhesive, in which case the dressing is released from the package by slitting or cutting the plastic films. Alternatively, the plastic films may be bonded together at least in part by means of a releasable adhesive (e.g. a pressure-sensitive adhesive contacting a silicone release layer on the opposite plastics sheet), so that the package can be opened by peeling apart the films. It may also be advantageous to provide the internal surfaces of the polymer films that contact the dressing with non-stick or release coatings to assist removal of the dressing from the package.

The present invention further provides method of manufacture of a cavity wound dressing according to the present invention, comprising the steps of: providing a microorganism-impermeable plastic sheet; providing one or more mold cavities in said plastic sheet for molding an elongated wound dressing; introducing a fluid precursor of a solid wound dressing material into said one or more mold cavities; and allowing said fluid precursor to set in said plurality of mold cavities.

Preferably, the step of providing the sheet or microorganism-impermeable material comprises providing first and second sheets of microorganism plastics material (which may be the same sheet folded over, or a opposite sides of a tubular sheet), and pressing the sheets together at intervals to define the molds. Alternatively, or additionally the molds may be provided by thermoforming mold cavities in one or both sheets.

Preferably, the precursor comprises an isocyanate capped polyurethane prepolymer as hereinbefore described.

Accordingly, in a further aspect the present invention provides a method of manufacture of a cavity wound dressing comprising the steps of: providing a two sheets of microorganism impermeable polymer film in face to face relationship with an elongate cavity therebetween; introducing a fluid precursor of a solid wound dressing material into said elongate cavity; and allowing the fluid precursor to set in the elongate cavity to provide an elongate cavity wound dressing in said cavity.

Preferably, the method further comprises sterilizing the wound dressing in the cavity without removing the wound dressing from the cavity. Preferably, the precursor comprises an isocyanate capped polyurethane prepolymer as hereinbefore described.

Preferably, the method according to the invention is adapted to produce a packaged wound dressing in accordance having one or more features or aspects of the present invention as hereinbefore described.

In another aspect, the present invention provides a method of making a packaged wound dressing in accordance with the invention, comprising the steps of: providing a plurality of molds; providing a filament, thread or string extending through the plurality of molds; introducing a fluid precursor of a solid wound dressing material into said plurality of molds; and allowing said fluid precursor to set in said plurality of molds.

It can thus be seen that the present invention provides an elongated absorbent cavity wound dressing that is packaged in contact with two opposed sheets of microorganism-impermeable plastic sheet material.

In other words, the invention provides an elongated absorbent cavity wound dressing that is encapsulated between two opposed sheets of microorganism-impermeable plastic sheet material wherein the opposed sheets contact the dressing to form a microorganism-impermeable barrier around the circumference of the dressing in at least a region of the dressing.

It will be appreciated that the medicinal articles according to the present invention have many uses, not only for filling and treatment of deep wounds, but also for insertion into and removal from other body cavities such as the mouth, gastrointestinal tract and vagina for therapeutic and prophylactic purposes.

The articles according to the invention are especially suitable for use in the treatment of deep and exuding wounds such as decubitis ulcers, pressure sores, venous ulcers, deep and open wounds and burns.

Specific examples of the present invention will now be described further by way of example with reference to the accompanying drawings, in which:

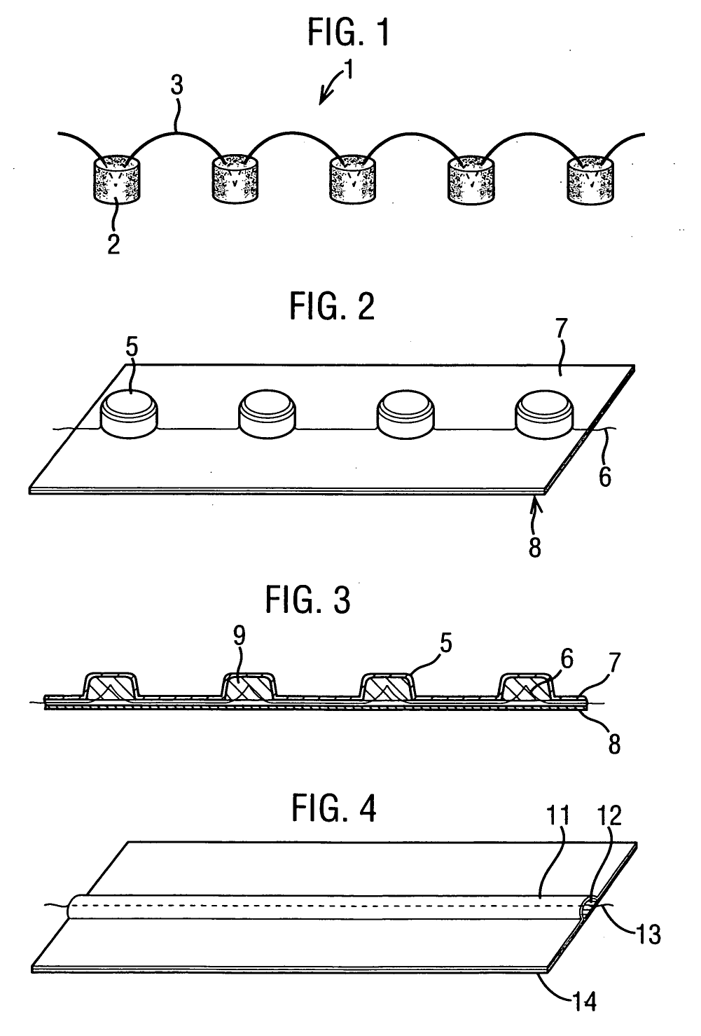

FIG. 1 shows a perspective view of a cavity wound dressing formed of linked absorbent bodies;

FIG. 2 shows a perspective view of a packaged cavity wound dressing according to the present invention;

FIG. 3 shows a longitudinal cross-section through the embodiment of FIG. 2; and

FIG. 4 shows a perspective view of a second packaged cavity wound dressing according to the present invention.

### EXAMPLE 1 (REFERENCE EXAMPLE)

A row of cylindrical molds of diameter 7 mm and depth 10 mm was drilled in a block of polytetrafluoroethylene (PTFE). Nylon threads were passed between each adjacent pair of molds, with the ends of each thread dipping into the molds. A hydrogel formulation was made from the following components: 25 g of HYPOL PreMA G60 (Registered Trade Mark) supplied by Dow Corning Ltd. and 100 g of water. The components were mixed and filled into the molds, ensuring that the ends of the nylon thread were well wetted with the mixture. The hydrogel was cured in an oven at 100° C. for about 15 minutes.

The resulting string of hydropolymer absorbent bodies was then stripped from the molds, and is shown in FIG. 1. It can be seen that the cavity dressing 1 comprises a number of polyurethane hydrogel beads 2 of substantially cylindrical shape, diameter about 8 mm and length about 8 mm. The beads are joined together by a medical grade nylon thread 3 that is embedded in each bead.

### EXAMPLE 2

A tube of polyethylene film of diameter about 2 cm was provided. A medical grade nylon thread was passed down the tube with the ends of the thread extending out of the ends of the tube. The tube was flattened slightly, and then filled with the a polyurethane foam forming composition comprising:

| | |
|---|---|
| HYPOL Prema G60 | 50 g |
| Water | 32 g |
| Acrylic copolymer | 12 g |
| Methanol | 6 g |

The acrylic copolymer is PRIMAL B-15J or RHOPLEX N-560 (Registered Trade Marks).

The tube was then immediately (i.e. before the foaming PU composition had set) clamped at intervals of about 1 cm along its length, thereby dividing the tube into compartments about 1 cm wide each separated by about 1 cm. The foamable composition was then allowed to cure to provide a string of polyurethane foam absorbent bodies joined by the nylon thread. The bodies are hygienically encapsulated in the polymer film tube, and may be sterilized in situ in the tube, for example by gamma-irradiation.

### EXAMPLE 3

A row of cylindrical recesses of diameter about 10 mm and depth about 5 mm were thermoformed in a strip of polyethylene film, the recesses being spaced apart by about 2.5 cm center to center. A silicone release coating was applied to the surface of the film and the recesses. A polyurethane foam forming composition as described in Example 2 was introduced into each of the recesses, and a nylon thread was then passed between each adjacent pair of molds with insertion of the thread into the foam forming composition in each recess. The composition was allowed to foam, cured and dried. A microorganism-impermeable polyethylene cover sheet coated with a medical grade pressure sensitive adhesive was then applied over the lower surface of the thermoformed sheet covering the thread and the recesses to enclose the recesses and the nylon thread in microorganism impermeable fashion.

The resulting encapsulated wound dressing is shown in FIG. 2. The upper sheet 7 may be formed from clear plastic to show the foamed disks therein. The nylon thread 6 is embedded in the disks to link the disks. The dressing can be manufactured continuously in lengths of 1 m, 5 m, 10 m or more, and the care giver can simply cut a length of the packaged dressing appropriate to the wound cavity being treated. The dressing is preferably sterilised, for example by gamma irradiation, and it is a further advantage that no secondary packaging is needed in order to maintain the sterility of the dressing.

### EXAMPLE 4

A strip of release-coated polyethylene film is provided having a longitudinal recessed channel of substantially semicircular cross-section and radius approximately 3 mm thermoformed along its length. A foamable polyurethane composition and a nylon thread are inserted into the channel in similar fashion to the procedure of Example 3. The composition is foamed, cured and dried, and a cover sheet is applied over the top of the film and channel again in similar fashion to Example 3.

The resulting packaged dressing is shown in FIG. 4. The channel 11 is substantially filled with the resilient hydrophilic polyurethane foam 12, through which runs the nylon thread 13. The cover sheet 14 can be removed by peeling, but provides a microorganism impermeable package for the cavity dressing and thereby removes any need for secondary packaging. The dressing is sterilised by gamma irradiation. The cover sheet and polyethylene film can be stripped from the dressing by the care giver immediately before use. A further advantage is that the dressing can be manufactured continuously in indefinite lengths. The care giver can simply cut an appropriate length for treatment of each particular cavity wound.

### EXAMPLE 5

The procedure of Example 4 is repeated with no thread extending along the tube. This results in an elongated slightly flattened resilient absorbent body of indefinite length encapsulated between the polymer sheets.

The above embodiments have been described by way of example only. Many other embodiments falling within the scope of the accompanying claims will be apparent to the skilled reader.

1. A packaged wound dressing comprising an elongated, absorbent cavity wound dressing that is packaged in contact with two opposed sheets of microorganism-impermeable plastic sheet material, wherein the opposed sheets contact the dressing to form a microorganism-impermeable barrier around the circumference of the dressing at least at intervals along the length of the dressing, whereby lengths can be cut from the dressing without the remainder of the dressing becoming contaminated.

2. A packaged wound dressing according to claim 1, wherein the absorbent dressing comprises a hydrophilic foam or a hydrogel.

3. A packaged wound dressing according to claim 1 or 2, comprising a plurality of absorbent bodies linked by one or more flexible bodies.

4. A packaged wound dressing according to claim 3, wherein the plurality of absorbent bodies comprises at least five absorbent bodies.

5. A packaged wound dressing according to claim 3 or 4, wherein the absorbent bodies each have a volume of from 0.001 to 10 cm³.

6. A packaged wound dressing according to claim 5, wherein the absorbent bodies each have a volume of from 0.01 to 1 cm³.

7. A packaged wound dressing according to any of claims 3 to 6, wherein the one or more flexible bodies comprise a filament, a thread or a string.

8. A packaged wound dressing according to claim 7, wherein the absorbent bodies are spaced along the filament, thread or string.

9. A packaged wound dressing according to claim 7 or 8, wherein the filament, thread or string extends through the absorbent bodies.

10. A packaged wound dressing according to any preceding claim, wherein the plastic films are bonded together at least in part by means of a releasable adhesive so that the package can be opened by peeling apart the films.

11. A packaged wound dressing according to any preceding claim, wherein an internal surface of the polymer films that contacts the dressing comprises a non-stick or release material to assist removal of the dressing from the package.

12. A packaged wound dressing according to any preceding claim, wherein the absorbent body comprises a single elongated absorbent body that extends the length of the dressing.

13. A packaged wound dressing according to any preceding claim, wherein the opposed sheets contact the dressing to form a microorganism-impermeable barrier around the circumference of the dressing substantially along the whole length of the dressing.

14. A packaged wound dressing according to any of claims 1 to 12, wherein the opposed sheets contact the dressing to form a microorganism-impermeable barrier around the circumference of the dressing at a plurality of intervals along the length of the dressing.

15. A method of making a packaged wound dressing as defined in any preceding claim, said method comprising the steps of:
providing a microorganism-impermeable plastic sheet;
providing one or more mold cavities in said plastic sheet for molding an elongated wound dressing;
introducing a fluid precursor of a solid wound dressing material into said one or more mold cavities; and
allowing said fluid precursor to set in said plurality of mold cavities.

16. A method according to claim 15, wherein the step of providing the mold cavities comprises providing first and second sheets of plastics material in face to face relationship, optionally with an elongated filament, thread or string extending between the sheets, and pressing the sheets together at intervals to define said molds.

17. A method according to claim 15, comprising the steps of: providing a two sheets of microorganism impermeable polymer film in face to face relationship with an elongated cavity therebetween; introducing a fluid precursor of a solid wound dressing material into said elongated cavity; allowing the fluid precursor to set in the elongated cavity to provide an elongated cavity wound dressing in said cavity.

18. A method according to any one of claims 15 to 17, wherein said precursor comprises an isocyanate capped polyurethane prepolymer.

19. A method according to any one of claims 15 to 18, further comprising the step of sterilizing the cavity wound dressing while it is encapsulated between said microorganism impermeable sheets.

20. A method according to any one of claims 15 to 19, wherein said method is carried out in continuous fashion to manufacture indefinite lengths of said packaged wound dressing.
